(19) United States
(12) Patent Application Publication
(10) Pub. No.: US 2024/0010680 A1
(43) Pub. Date: Jan. 11, 2024

(54) PEPTIDE DISINFECTANT FOR PREVENTING CORONAVIRUSES

(71) Applicant: Xiamen University, Xiamen (CN)

(72) Inventors: Chimeng TZENG, Xiamen (CN); Minhuang HU, Xiamen (CN); Xueting WANG, Xiamen (CN); Bingyi ZHENG, Xiamen (CN); Caiying ZENG, Xiamen (CN); Xiaoling ZENG, Xiamen (CN)

(21) Appl. No.: 18/350,803

(22) Filed: Jul. 12, 2023

Related U.S. Application Data
(63) Continuation of application No. PCT/CN22/71161, filed on Jan. 11, 2022.

(30) Foreign Application Priority Data
Jan. 12, 2021 (CN) 202110034714.4

(51) Int. Cl. C07K 7/06; A01N 25/00; A01P 1/00

(52) U.S. Cl. CPC C07K 7/06; A01N 25/00; A01P 1/00

(57) ABSTRACT

A peptide disinfectant for preventing coronaviruses is provided. The peptide disinfectant for preventing coronaviruses comprises an active ingredient of the peptide disinfectant comprises mixed peptides comprising a first peptide (SEQ ID NO. 01), a second peptide (SEQ ID NO. 02), and a third peptide (SEQ ID NO. 03).

### RELATED APPLICATIONS

This application a continuation of International patent application PCT/CN2022/071161, filed on Jan. 11, 2022, which claims priority to Chinese patent application 202110034714.4, filed on Jan. 12, 2021. International patent application PCT/CN2022/071161 and Chinese patent application 202110034714.4 are incorporated herein by reference.

### REFERENCE TO AN ELECTRONIC SEQUENCE LISTING

The contents of the electronic sequence listing (SequenceListing.xml; Size: 10,873 bytes; and Date of Creation: Jul. 11, 2023) is herein incorporated by reference.

### FIELD OF THE DISCLOSURE

The present disclosure relates to the technical field of antivirus agents, and in particular relates to a peptide disinfectant for preventing coronaviruses.

### BACKGROUND OF THE DISCLOSURE

Viruses of Coronaviridae are ribonucleic acid (RNA) viruses with capsids and linear positive-sense single-strand genomes and are a large group of viruses widely existing in nature. Coronaviruses capable of infecting humans are known as follows: HCoV-229E, HCoV-0C43, HCoV-NL63, HCoV-HKU1, SARS-CoV, MERS-CoV, and 2019-nCOV, in which SARS-CoV, MERS-CoV, and 2019-nCoV are β-coronaviruses and highly infectious, causing a great risk to human health and serious economic damage. At the same time, an alternative emergence of different types of coronaviruses is expected within the next few decades according to an emergence pattern of the existing coronaviruses, thus seriously affecting the development of human society. Given that there is currently no specific drug for the coronavirus, a search for a more effective prevention method is undoubtedly the best way to deal with the coronaviruses.

### BRIEF SUMMARY OF THE DISCLOSURE

The present disclosure provides a peptide disinfectant for preventing coronaviruses to solve the existing technique deficiencies.

A technical solution of the present disclosure is as follows:

A peptide disinfectant for preventing coronaviruses, an active ingredient of the peptide disinfectant comprises mixed peptides comprising a first peptide (SEQ ID NO. 01), a second peptide (SEQ ID NO. 02), and a third peptide (SEQ ID NO. 03), a concentration of the first peptide is 21-22 μmol/L (μM), a concentration of the second peptide is 25-26 μM, and a concentration of the third peptide is 36-37 μM.

In a preferred embodiment of the present disclosure, the concentration of the first peptide is 21.38 μM.

In a preferred embodiment of the present disclosure, the concentration of the second peptide is 25.30 μM.

In a preferred embodiment of the present disclosure, the concentration of the third peptide is 36.82 μM.

In a preferred embodiment of the present disclosure, the peptide disinfectant further comprises honeysuckle, menthol, Natural Indigo, boric acid, glycerin, and water.

Another technical solution of the present disclosure is as follows:

A peptide disinfectant for preventing coronaviruses, an active ingredient of the peptide disinfectant comprises mixed peptides comprising a first peptide (SEQ ID NO. 01) and a third peptide (SEQ ID NO. 03), a concentration of the first peptide is 21-22 μmol/L (μM), and a concentration of the third peptide is 36-37 μM.

In a preferred embodiment of the present disclosure, the concentration of the first peptide is 21.38 μM.

In a preferred embodiment of the present disclosure, the concentration of the third peptide is 36.82 μM.

In a preferred embodiment of the present disclosure, the concentration of the first peptide is 21.38 μM, and the concentration of the third peptide is 36.82 μM.

In a preferred embodiment of the present disclosure, the peptide disinfectant further comprises honeysuckle, menthol, Natural Indigo, boric acid, glycerin, and water.

The present disclosure has the following advantages.
1. The components of the mixed peptide of the present disclosure are all small peptide molecules with six amino acid residues prone to be prepared and synthesized with low costs and wide applications, and coronaviruses are effectively prevented.
2. The excipients of the present disclosure are simple and easily obtained, so that the present disclosure has efficient preventive effects as well as good use values for promotion.

### BRIEF DESCRIPTION OF THE DRAWING

FIG. 1 illustrates a graph of binding capacity between Peptide1 and hACE2 in Embodiment 1 of the present disclosure.

FIG. 2 illustrates a graph of binding capacity between Peptide2 and the hACE2 in Embodiment 1 of the present disclosure.

FIG. 3 illustrates a graph of results of an in-vitro binding test between hACE2-His and S1-HA purified protein of Embodiment 2 of the present disclosure.

### DETAILED DESCRIPTION OF THE EMBODIMENTS

The technical solutions of the present disclosure are further described below in combination with the accompanying drawings and embodiments.

### Embodiment 1: Testing a Binding Capacity Between Peptide1 & Peptide2 and Derivatives Thereof and hACE2 Based on a Biacore Molecular Binding System (e.g., GE Biacore T200)

Apparatus: GE Biacore T200.

Materials and reagents: Series S Sensor Chip CM5 (GE Life100530), hACE2 purified protein, Peptide1 & Peptide2 and their derivatives (See Table 1), Amine Coupling Kit (GELife100050, Phosphate-buffered saline (PBS), glycine-hydrochloric acid buffer solution (pH 2.5)).

TABLE 1

| Names of | Amino acid | Sequence |
|---|---|---|
| peptides | sequence | number |
| Peptide1 | YKYRYL | SEQ ID NO. 04 |
| Peptide11 | YRYKYL | SEQ ID NO. 05 |
| Peptide12 | YKYKYL | SEQ ID NO. 01 |
| Peptide13 | YRYRYL | SEQ ID NO. 02 |
| Peptide2 | YSYINK | SEQ ID NO. 06 |
| Peptide21 | YSYIQK | SEQ ID NO. 03 |
| Peptide22 | YTYINK | SEQ ID NO. 07 |
| Peptide23 | YTYIQK | SEQ ID NO. 08 |

Method of Experiment:

(1) Preparing a Capture Surface and a Binding Experiment

Materials: 1-(3-Dimethylaminopropyl)-3-ethylcarbodiimide hydrochloride (EDC), N-Hydroxysuccinimide (NHS), ethanolamine (an amine coupling kit), PBS, hACE2, Series S Sensor Chip CM5.

Specific Steps:
a. The GE Biacore T200 is started, the Series S Sensor Chip CM5 is embedded into the GE Biacore T200, the PBS is degassed to obtain a degassed PBS, and the degassed PBS is used throughout the experiment;
b. The NHS, the EDC, the ethanolamine, and the hACE2 (1 mg/mL) are placed on a feeding plate of the GE Biacore T200 according to volumes indicated by the GE Biacore T200 using Eppendorf (EP) tubes in sufficient quantities;
c. A sample is injected at a flow rate of 5 μL/min, an activation of the Series S Sensor Chip CM5, amine coupling of the hACE2, and saturating an activated carboxyl group that is uncoupled with the hACE2 using the ethanolamine are sequentially performed to obtain an RUs value of the hACE2, and the RUs value is 14826.0;
d. A mobile solution (the degassed PBS) is closed, a command window is closed, and a report is served to obtain a CM5-hACE2 chip;

(2) Affinity analysis of peptides with hACE from Table 1

Materials: the PBS, the CM5-hACE2 chip, the peptides in the Table 1, and the glycine-hydrochloric acid buffer solution (pH 2.5).

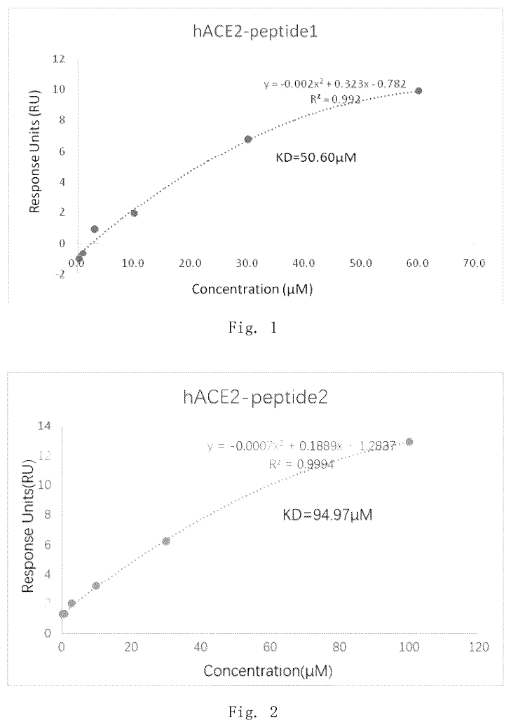

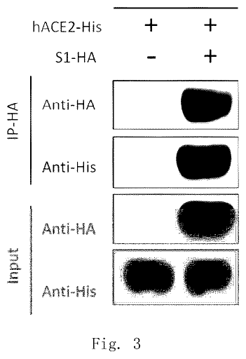

Specific Steps:
a. Preparing different dilutions as samples for testing: the peptides in Table 1 to be tested at a concentration of 3 mmol/L (mM) are dissolved and sequentially diluted into different dilutions of 100 μmol/L (μM), 60 μM, 30 μM, 10 μM, 3 μM, 1 μM, 0.3 μM, and 0 using the PBS to obtain the samples. Each of the different dilutions is ensured to have a volume of no less than 400 μL and separately packaged on EP tubes. The samples are placed on a test plate of the GE Biacore T200, and the test plate is stuck in a test slot;
b. The CM5-hACE2 chip is loaded, a test procedure is set as follows: an injection flow rate of the samples is set to be 30 μL/minute for 120 seconds, a dissociation time is set to be 300 seconds, the glycine-hydrochloric acid buffer solution is injected at a flow rate of 30 μL/minute for 30 seconds to regenerate the CM5-hACE2 chip, the samples with different concentrations are sequentially tested according to a same procedure, and RU values obtained from the samples with different concentrations are fitted to obtain an affinity constant (K D value) (Referring to Tables 2 and 3, the binding capacity between the Peptide1 and the hACE2 is shown in FIG. 1, and the binding capacity between the Peptide2 and the hACE2 is shown in FIG. 2).

TABLE 2

| | Amino acid | K_{D} value |
|---|---|---|
| Peptides | sequence | (μM) |
| Peptide1 | YKYRYL | 50.60 |
| Peptide11 | YRYKYL | 60.70 |
| Peptide12 | YKYKYL | 21.38 |
| Peptide13 | YRYRYL | 25.30 |
| Peptide2 | YSYINK | 94.97 |
| Peptide21 | YSYIQK | 36.82 |
| Peptide22 | YTYINK | 58.37 |
| Peptide23 | YTYIQK | 46.53 |

TABLE 3

| | Molecular | Equilibrium |
|---|---|---|
| | concentration | dissociation |
| Peptides | (μM) and ratios | constants (μM) |
| Peptide1 + Peptide2 | 50.60 μM:94.97 μM | 76.38 |
| Peptide1 + Peptide12 | 50.60 μM:21.38 μM | 19.36 |
| Peptide12 + Peptide13 | 21.38 μM:25.30 μM | 14.17 |
| Peptide1 + Peptide11 | 50.60 μM:60.70 μM | 35.86 |
| Peptide1 + Peptide21 | 50.60 μM:36.82 μM | 20.11 |
| Peptide12 + Peptide21 | 21.38 μM:36.82 μM | 8.13 |
| Peptide11 + Peptide22 + | 60.70 μM:58.37 | 65.13 |
| Peptide2 | μM:94.97 μM | |
| Peptide12 + Peptide13 + | 21.38 μM:25.30 | 5.21 |
| Peptide21 | μM:36.82 μM | |

### Embodiment 2: A Mechanism for Inhibiting S1 Protein from Binding to hACE2 by Peptide1 & Peptide2, Derivatives Thereof, and Peptides with Different Ratios is Described Using Immunoprecipitation (IP)

Apparatus: Westernblot electrophoresis equipment (BIO-RAD Mini-PROTEAN® Tetra).

Materials and reagents: hACE2-His purified protein, Peptide1& Peptide2 and derivatives thereof, S1-HA purified protein, the PBS, 5% bovine serum albumin (BSA) blocking solution, 1.5 mol (M) Tris (pH 8.8), 1.0 M Tris (pH 6.8), 10% sodium dodecyl sulfate (SDS), 10% ammonium persulphate, antibody eluate, diaminobenzidine (DAB) horseradish peroxidase colour development kit (beyotime P0202), SDS-Gly electrophoresis solution, electron-transfer solution, polyvinylidene fluoride (PVDF) membrane (Beyotime,FFP28), anti-6×His (Abcam, ab18184), anti-HA tag (Abcam, ab9110), HA-tag agarose beads (Engibody, AT0079-1 mL), and SDS-PAGE protein loading buffer solution (Beyotime, P0286-2 mL).

Method of Experiment:

(1) A Binding Test Between the hACE2-his Purified Protein and the S1-HA Purified Protein In Vitro

Materials: the PBS, the hACE2-His purified protein, the S1-HA purified protein, the HA-tag agarose beads, and the SDS-PAGE protein loading buffer.

Steps: two tubes of 30 μL of the HA-tag agarosebeads are separately packaged and placed on ice for use, two tubes of the hACE2-His purified protein are separately packaged with a specified volume, the S1-HA purified protein with the specified volume is added into one tube of the two tubes of the hACE2-His purified protein to obtain a first sample, the PBS with the specified volume is added into the other tube of the two tubes of the hACE2-His purified protein to obtain a second sample The first sample and the second sample are mixed separately and heated in a 37° C. water bath for 30 minutes, one-tenth of the first sample and one-tenth of the second sample are taken out and then frozen at −80° C. to use as an input sample. The rest of the first sample and the rest of the second sample are mixed with the two tubes of 30 μL of the HA-tag agarose beads, the PBS is added to replenish volumes of the first sample and the second sample to be 450 μL, and the IP is then performed overnight at 4° C. The HA-tag agarose beads are washed off free proteins with the PBS the next day, and 100 μL of the PBS and the SDS-PAGE protein loading buffer are respectively added to obtain IP samples. The IP samples are denatured by heating together with the input sample, and Western-blot test is performed. Contents of the hACE2-His purified protein and the S1-HA purified protein of control and experimental groups in the IP samples and the input sample are respectively tested using anti-His and anti-HA, as shown in FIG. 3.

(2) Testing a Binding Capacity Between the hACE2-his Purified Protein and the S1-HA Purified Protein after the Peptide1 & Peptide2 and Derivatives Thereof are Added to the IP Samples

Materials: the PBS, the hACE2-His purified protein, the S1-HA purified protein, the HA-tag agarose beads, the SDS-PAGE protein loading buffer solution, the peptide 1 & peptide2 and derivatives thereof.

Procedure: 10 tubes of the HA-tag agarose beads with specified amounts are separately packaged, washed with the PBS, and placed on ice. 10 tubes of the hACE2-His purified protein with specified amounts are then separately packaged, Peptide1: Peptide2 (50.601 μM: 94.971 μM), Peptide1: Peptide12 (50.601 μM: 21.38 μM), Peptide12: Peptide13 (21.38 μM: 25.30 μM), Peptide1: Peptide11 (50.60 μM: 60.70 μM), Peptide1: Peptide21 (50.60 μM: 36.82 μM), Peptide12: Peptide21 (21.38 μM: 36.82 μM), Peptide11: Peptide22: Peptide2 (60.70 μM: 58.37 μM: 94.97 μM), Peptide12: Peptide13: Peptide21 (21.38 μM: 25.301 μM: 36.82 μM) are separately added and mixed to even. The PBS is added into 1 tube of the 10 tubes of the hACE2-His purified protein to use as a blank group, and no peptides is not added into blank group. The S1-HA purified protein is added into the rest 9 tubes of the 10 tubes of the hACE2-His purified protein respectively, and the rest 9 tubes of the hACE2-His purified protein heated in a 37° C. water bath for 30 minutes to use as experimental groups and a positive control group. No peptides is not added into the positive control group. One-tenth of mixtures of the control group and the experimental groups are taken out and then frozen at −80° C. to use as input samples, and remaining components of the mixtures are mixed with the HA-tag agarose beads. The PBS is added to replenish volumes of the blank group, the experimental groups, and the positive control group to be 450 μL, and the IP is then performed overnight at 4° C. to obtain IP samples, Westernblot test is performed the next day to test contents of the hACE2-His purified protein and the S1-HA purified protein of the input samples and the IP samples in each group of the blank group, the experimental groups and the positive control group. A corresponding relative brightness value in the blank group is subtracted from relative brightness values of the hACE2-His purified protein in the experimental groups and the positive control group, and relative brightness values of the experimental groups are then compared to a relative brightness value of the positive control group to finally obtain an inhibition capacity relative to a binding between the hACE2-His purified protein and the S1-HA purified protein after a combination of the peptides in each of the experimental groups, as shown in Table 4.

TABLE 4

| | | Relative |
|---|---|---|
| | concentration (μM) | brightness |
| Peptides | and ratios | value |
| Peptide1:Peptide2 | 50.60 μM:94.97 μM | 45.32% |
| Peptide1:Peptide12 | 50.60 μM:21.38 μM | 32.51% |
| Peptide12:Peptide13 | 21.38 μM:25.30 μM | 24.73% |
| Peptide1:Peptide11 | 50.60 μM:60.70 μM | 39.65% |
| Peptide1:Peptide21 | 50.60 μM:36.82 μM | 35.57% |
| Peptide12:Peptide21 | 21.38 μM:36.82 μM | 28.66% |
| Peptide11:Peptide22:Peptide2 | 60.70 μM:58.37 μM:94.97 | 18.15% |
| | μM | |
| Peptide12:Peptide13:Peptide21 | 21.38 μM:25.30 μM:36.82 | 7.13% |
| | μM | |
| S1-HA purified protein | — | 100% |

### Embodiment 3

A peptide disinfectant for preventing coronaviruses comprises a mixed peptide, honeysuckle, menthol, Natural Indigo, boric acid, glycerol, and water for injection, wherein the mixed peptide comprises a first peptide 12, a second peptide 13, and a third peptide 21. A concentration of the first peptide 12 is 21.38 μM, a concentration of the second peptide 13 is 25.30 μM, and a concentration of the third peptide 21 is 36.82 μM. A concentration of the honeysuckle is 17 wt %, a concentration of the menthol is 1.0 wt %, a concentration of the Natural Indigo is 2 wt %, a concentration of the boric acid is 0.15 wt %, a concentration of the glycerol is 15 wt %, and the balance is water.

A method for preparing the peptide disinfectant is as follows: fully dissolving the honeysuckle, the menthol, the Natural Indigo, the boric acid, and the glycerol in the water for injection to configure an auxiliary mother liquid, dissolving the mixed peptides in the auxiliary mother solution, and finally replenishing with the water for injection.

### Embodiment 4

A peptide disinfectant for preventing coronaviruses comprises a mixed peptide, honeysuckle, menthol, Natural Indigo, boric acid, glycerol, and water for injection, wherein the mixed peptide comprises a first peptide 12 and a third peptide 21. A concentration of the first peptide 12 is 21.38 μM, and a concentration of the third peptide 21 is 36.82 μM.

A concentration of the honeysuckle is 17 wt %, a concentration of the menthol is 1.0 wt %, a concentration of the Natural Indigo is 2 wt %, a concentration of the boric acid is 0.15 wt %, a concentration of the glycerol is 15 wt %, and the balance is water.

A method for preparing the peptide disinfectant is as follows: fully dissolving the honeysuckle, the menthol, the Natural Indigo, the boric acid, and the glycerol in the water for injection to configure an auxiliary mother liquid, dissolving the mixed peptides in the auxiliary mother solution, and finally replenishing with the water for injection.

The above mentioned is only one example of the present disclosure, so it cannot be used to limit the scope of the present disclosure, i.e. the equivalent changes and modifications made according to the scope of the patent and the specification of the present disclosure should still fall within the scope of the present disclosure.

What is claimed is:

1. A peptide disinfectant for preventing coronaviruses, wherein:
an active ingredient of the peptide disinfectant comprises mixed peptides comprising a first peptide (SEQ ID NO. 01), a second peptide (SEQ ID NO. 02), and a third peptide (SEQ ID NO. 03),
a concentration of the first peptide is 21-22 μmol/L (μM),
a concentration of the second peptide is 25-26 μM, and
a concentration of the third peptide is 36-37 μM.

2. The peptide disinfectant according to claim 1, wherein the concentration of the first peptide is 21.38 μM.

3. The peptide disinfectant according to claim 1, wherein the concentration of the second peptide is 25.30 μM.

4. The peptide disinfectant according to claim 1, wherein the concentration of the third peptide is 36.82 μM.

5. The peptide disinfectant according to claim 1, wherein the peptide disinfectant further comprises honeysuckle, menthol, Natural Indigo, boric acid, glycerin, and water.

6. A peptide disinfectant for preventing coronaviruses, wherein:
an active ingredient of the peptide disinfectant comprises mixed peptides comprising a first peptide (SEQ ID NO. 01) and a third peptide (SEQ ID NO. 03),
a concentration of the first peptide is 21-22 μmol/L (μM), and
a concentration of the third peptide is 36-37 μM.

7. The peptide disinfectant according to claim 6, wherein the concentration of the first peptide is 21.38 μM.

8. The peptide disinfectant according to claim 6, wherein the concentration of the third peptide is 36.82 μM.

9. The peptide disinfectant according to claim 6, wherein:
the concentration of the first peptide is 21.38 μM, and
the concentration of the third peptide is 36.82 μM.

10. A peptide disinfectant according to claim 6, wherein the peptide disinfectant further comprises honeysuckle, menthol, Natural Indigo, boric acid, glycerin, and water.

11. The peptide disinfectant according to claim 2, wherein the peptide disinfectant further comprises honeysuckle, menthol, Natural Indigo, boric acid, glycerin, and water.

12. The peptide disinfectant according to claim 3, wherein the peptide disinfectant further comprises honeysuckle, menthol, Natural Indigo, boric acid, glycerin, and water.

13. The peptide disinfectant according to claim 4, wherein the peptide disinfectant further comprises honeysuckle, menthol, Natural Indigo, boric acid, glycerin, and water.

14. A peptide disinfectant according to claim 7, wherein the peptide disinfectant further comprises honeysuckle, menthol, Natural Indigo, boric acid, glycerin, and water.

15. A peptide disinfectant according to claim 8, wherein the peptide disinfectant further comprises honeysuckle, menthol, Natural Indigo, boric acid, glycerin, and water.

16. A peptide disinfectant according to claim 9, wherein the peptide disinfectant further comprises honeysuckle, menthol, Natural Indigo, boric acid, glycerin, and water.


SEQUENCE LISTING (8 sequences)

SEQ ID NO: 1 - protein, 6 aa, synthetic construct
  REGION 1..6: note = Synthetic
YKYKYL

SEQ ID NO: 2 - protein, 6 aa, synthetic construct
  REGION 1..6: note = Synthetic
YRYRYL

SEQ ID NO: 3 - protein, 6 aa, synthetic construct
  REGION 1..6: note = Synthetic
YSYIQK

SEQ ID NO: 4 - protein, 6 aa, synthetic construct
  REGION 1..6: note = Synthetic
YKYRYL

SEQ ID NO: 5 - protein, 6 aa, synthetic construct
  REGION 1..6: note = Synthetic
YRYKYL

SEQ ID NO: 6 - protein, 6 aa, synthetic construct
  REGION 1..6: note = Synthetic
YSYINK

SEQ ID NO: 7 - protein, 6 aa, synthetic construct
  REGION 1..6: note = Synthetic
YTYINK

SEQ ID NO: 8 - protein, 6 aa, synthetic construct
  REGION 1..6: note = Synthetic
YTYIQK